(19) United States
(12) Patent Application Publication
(10) Pub. No.: US 2014/0121698 A1
(43) Pub. Date: May 1, 2014

(54) SYSTEM FOR STORING AND DISPENSING MULTIPLE FLUIDS AND A METHOD FOR USING THE SAME

(71) Applicant: NORDSON CORPORATION, Westlake, OH (US)

(72) Inventors: Thomas A. Kirk, Hastings, MN (US); Todd W. Sharratt, Stillwater, MN (US)

(73) Assignee: NORDSON CORPORATION, Westlake, OH (US)

(21) Appl. No.: 13/777,507

(22) Filed: Feb. 26, 2013

Related U.S. Application Data
(60) Provisional application No. 61/719,991, filed on Oct. 30, 2012.

(51) Int. Cl. A61B 17/00

(52) U.S. Cl. CPC A61B 17/0057
USPC 606/213

(57) ABSTRACT

A system for storing and dispensing first and second fluids, comprising a cartridge, further comprising a barrel having a wall, a proximal end and a distal end. A first piston is disposed within the wall, extending distally from the proximal end. A delivery tube is disposed within the wall and fluidicly communicates with a first outlet. A first fluid chamber is disposed between the first piston and the delivery tube and fluidicly communicates with the first outlet. A second piston is disposed between the wall and the delivery tube. A second fluid chamber is between the second piston and the distal end, the second fluid chamber fluidity communicating with a second outlet. A transmission structure is operative to transmit force from the first piston to the second piston to thereby dispense the first and second fluids from the first and second outlets.

This application claims the priority of U.S. Provisional Patent Application Ser. No. 61/719,991, filed on Oct. 30, 2012 (pending), the disclosure of which is incorporated by reference herein.

### BACKGROUND OF THE INVENTION

Generally, it is well known to dispense multiple fluids from a syringe or other device. There are problems that arise when using the systems in the prior art. Due to the nature of a syringe, and especially syringes having multiple fluid chambers, pressure changes occur within the syringe when one or more components move relative to other components within the body of the syringe. Disadvantageously, negative pressure, or a vacuum, can form within the body of the syringe barrel, potentially causing problems with the dispensing of the fluids from the syringe.

Moreover, it is often desired to attach a syringe to a downstream device such as tubing at the syringe outlet. With syringes having multiple outlets, crowding at the outlets becomes a problem when the outlets are connected with tubing and other downstream devices.

There is a need for an apparatus and method of dispensing multiple fluids and counteracting pressure changes that enables the connection of downstream devices to the system that addresses present challenges and characteristics such as those discussed above.

### SUMMARY

The present invention provides a system for storing and dispensing first and second fluids comprising a cartridge. The cartridge further comprises a barrel having a wall, a proximal end and a distal end. A first piston is disposed within the wall and extends distally from the proximal end. A delivery tube is disposed within the wall and fluidicly communicates with a first outlet. A first fluid chamber is disposed between the first piston and delivery tube and fluidicly communicates with the first outlet. A second piston is disposed between the wall and the delivery tube. A second fluid chamber is between the second piston and the distal end and fluidicly communicates with a second outlet. The cartridge further comprises a transmission structure operative to transmit force from the second piston to thereby dispense the first and second fluids from the first and second outlets. The system also comprises a cannula assembly coupled with and fluidicly communicating with the first and second outlets. The cannula assembly further comprises a tip at a distal portion of the cannula assembly. The tip is adapted to dispense the first and second fluids from the cannula assembly. In a preferred embodiment, the distal end of the barrel further comprises a tip adaptor. At least part of the second outlet is disposed within and extends distally from the tip adaptor. In one embodiment, at least one of the first or second outlets is disposed radially outward of a central axis of the barrel. In one embodiment, the transmission structure is a plunger. In an alternative embodiment, the transmission structure is pneumatic.

The present invention also provides a cartridge for storing and dispensing first and second fluids comprising a barrel having a wall, a proximal end and a distal end. The cartridge further comprises a first piston disposed within the wall and extending distally from the proximal end. A delivery tube is also provided. The delivery tube is disposed within the wall and fluidicly communicates with a first outlet. A first fluid chamber is disposed between the first piston and the delivery tube and fluidicly communicates with the first outlet. A second piston is disposed between the wall and the delivery tube. A second fluid chamber is between the second piston and the distal end and fluidicly communicates with a second outlet. A vent is provided and is operative to prevent a vacuum from forming within the barrel. At least a part of the vent is disposed between the first piston and the wall.

The present invention also provides a method of counteracting pressure changes within a cartridge for storing and dispensing fluids. The method comprises advancing a plunger within a barrel. Advancing the plunger causes a movement of at least a first internal component in the barrel relative to at least a second internal component. The method further comprises forming a cavity therebetween and fluidicly communicating the cavity with a space outside the cartridge.

A method of dispensing multiple fluids from a storage and dispensing system is provided. The system includes a barrel, first and second pistons, first and second fluid chambers containing first and second fluids, a transmission structure, and first and second outlets. The method includes fluidicly communicating the first and second outlets with cannula assembly.

The method further includes advancing the transmission structure from a proximal end of the barrel towards a distal end, thereby advancing the first and second pistons within the barrel and discharging the first and second fluids from the first and second fluid chambers, through the first and second outlets and into the first and second lumens of the first and second tubes, and out of first and second apertures.

The present invention also provides a method of dispensing first and second biomaterial fluids from a cartridge. The cartridge includes a barrel having a wall, a proximal end and a distal end, and first and second chambers axially disposed relative to one another. The method comprises the steps of moving a first piston disposed within the wall of the barrel towards the distal end. The distal movement of the first piston causes the first biomaterial fluid to leave the first chamber and enter into a first channel and out a first outlet. The distal movement of the first piston also causes distal movement of the second piston, thereby causing the second biomaterial fluid to leave the second chamber and enter into a second channel and out of a second outlet. The first biomaterial fluid thereby passes from the first outlet to a first lumen of a cannula assembly and the second biomaterial fluid passes from the second outlet to a second lumen of the cannula assembly. The method further comprises dispensing the first and second fluids from the cannula assembly by the aid of a pressurized gas. The method further comprises mixing the first and second fluids in flight to form a mixture and depositing the mixture onto a portion of a human body.

### BRIEF DESCRIPTION OF THE DRAWINGS

FIG. 1 is a perspective view of a system for storing and dispensing multiple fluids.

FIG. 2A is a cross-sectional side view of a system for storing and dispensing multiple fluids in a filled position.

FIG. 2B is a cross-sectional side view of a system for storing and dispensing multiple fluids as the fluids are being drawn out of the system.

FIG. 2C is a cross-sectional side view of a system for storing and dispensing multiple fluids in a generally empty position.

FIG. 3A is a detailed cross-sectional side view of a system for storing and dispensing multiple fluids showing a vent.

FIG. 3B is a detailed cross-sectional side view of a system for storing and dispensing multiple fluids showing a vent fluidicly communicating with a cavity.

FIG. 4 is a perspective view of alternative embodiment of the system of FIG. 1 being pneumatically driven.

FIG. 5A is a cross-sectional side view of the system of FIG. 4 in a filled position.

FIG. 5B is a cross-sectional side view of the system of FIG. 4 in a generally empty position.

FIG. 6A is a cross-sectional side view of an alternative embodiment of a system for storing and dispensing multiple fluids in a filled position.

FIG. 6B is a cross-sectional side view of an alternative embodiment of a system for storing and dispensing multiple fluids in a generally empty position.

### DETAILED DESCRIPTION

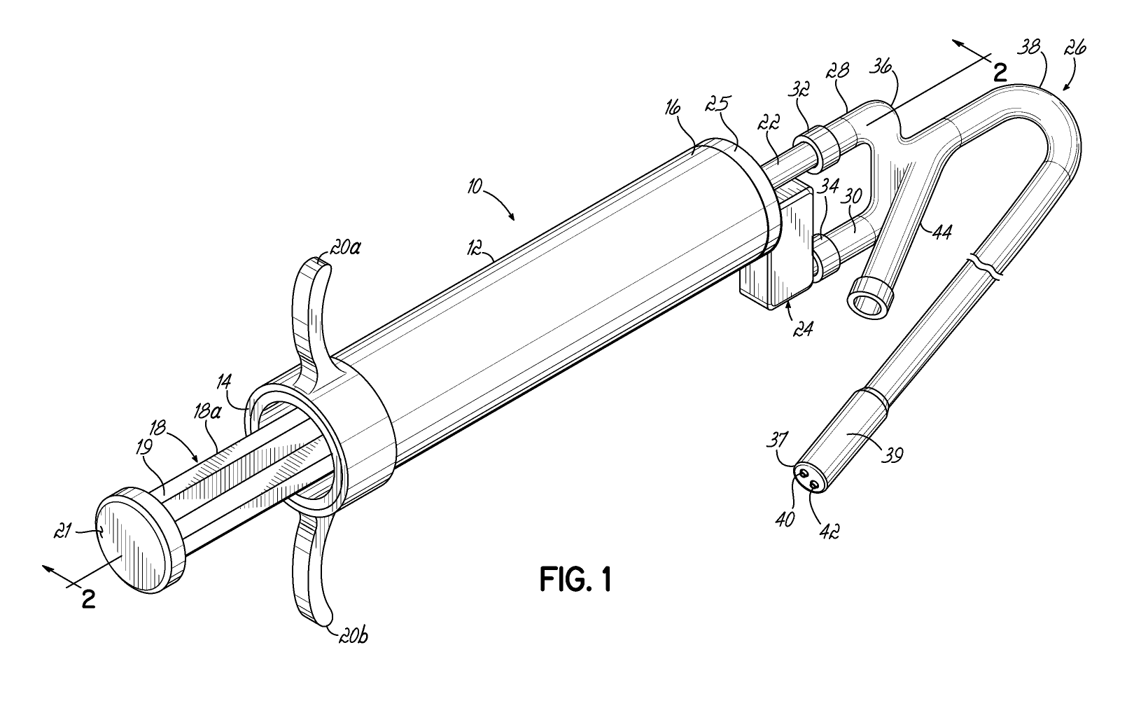

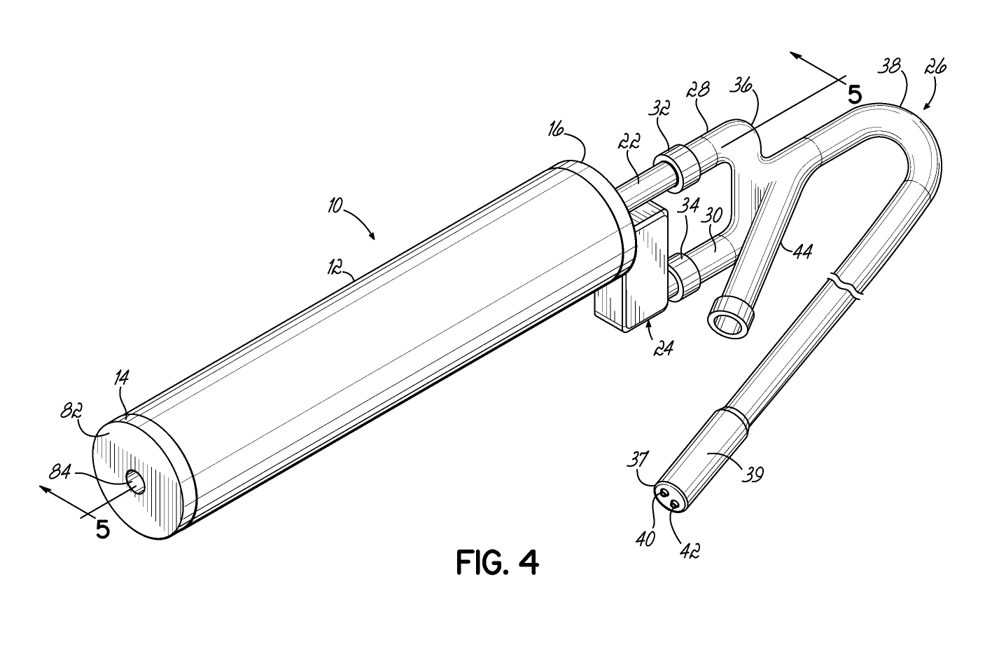

FIG. 1 shows a preferred embodiment of the system. The system comprises a cartridge 10 including a barrel 12, the barrel 12 having a proximal end 14, a distal end 16 and a generally cylindrical shape. The proximal end 14 is open to accept a plunger or transmission structure 18. The transmission structure 18 preferably has a shaft 19 and a depressing surface 21 against which a user presses a thumb or other finger to advance the transmission structure 18 distally. In an alternative embodiment as seen in FIG. 4, the cartridge 10 includes a pneumatic adaptor 82 adapted to advance the internal components of the barrel 12 instead of a plunger 18a. The barrel 12 preferably has finger grips 20a, 20b extending radially therefrom in order to assist the user in depressing the plunger 18a. In a preferred embodiment, the finger grips 20a, 20b comprise two members extending radially from the barrel 12. However, the finger grips 20a, 20b may alternatively comprise a radially extending flange or another configuration that assists the user in depressing the plunger 18a.

The distal end 16 of the barrel 12 has first and second outlets 22, 24 extending therefrom. The first and second outlets 22, 24 are fluidicly connected to a cannula assembly 26. Specifically, the first outlet 22 is connected to a first tube 28 and the second outlet 24 is connected to a second tube 30 of the cannula assembly 26. In a preferred embodiment, a portion of the second outlet 24 extends radially from the first outlet 22 in order to provide ample space for the connection with the first and second tubes 28, 30 of the cannula assembly 26. The first and second tubes 28, 30 each include adaptors 32, 34 configured to accept the first and second outlets 22, 24 and provide a seal between the first and second outlets 22, 24 and the first and second tubes 28, 30, respectively. There are first and second lumens (not shown) within the first and second tubes 28, 30, respectively. At the proximal end 36 of the cannula assembly 26, the first and second tubes 28, 30 bifurcate in order to facilitate a connection with the first and second outlets 22, 24. More distally along the cannula assembly 26, the first and second tubes 28, 30 converge into a main cannula 38. Within the main cannula 38 the first and second fluids that are discharged from the first and second outlets 22, 24 remain separated in first and second lumens (not shown) within the main cannula 38. The first and second lumens are fluidicly connected with the first and second tubes 28, 30. When the transmission structure 18 is depressed distally, the first and second fluids are discharged from the first and second outlets 22, 24, into and through the first and second tubes 28, 30 and into the first and second lumens. The first and second fluids may be, for example, biomedical materials used in a medical procedure. The first and second fluids travel within the first and second lumens within the main cannula 38 until reaching the tip 39 at the distal end 37 of the cannula assembly 26, whereupon the first and second fluids are discharged from first and second apertures 40, 42 of the tip 39. Extending radially from the main cannula 38 is an additional tube 44 adapted to communicate with a gas source. In a preferred embodiment, the first aperture 40 is situated at least partially transverse to the second aperture 42, whereby the first and second fluids interact after being dispersed from the first and second apertures 40, 42. Preferably, an interaction between the first and second fluids creates a third material comprising the first and second fluids.

Due to the configuration of tube 44 and cannula assembly 26, the system is operative to dispense the first and second fluids from the first and second apertures 40, 42 by the aid of a pressurized gas. In a preferred embodiment, the first and second fluids are mixed in flight after being dispensed with the aid of a pressurized gas from the first and second apertures 40, 42.

The mixture of the first and second fluids is deposited onto a biological substrate such as a portion of the human body.

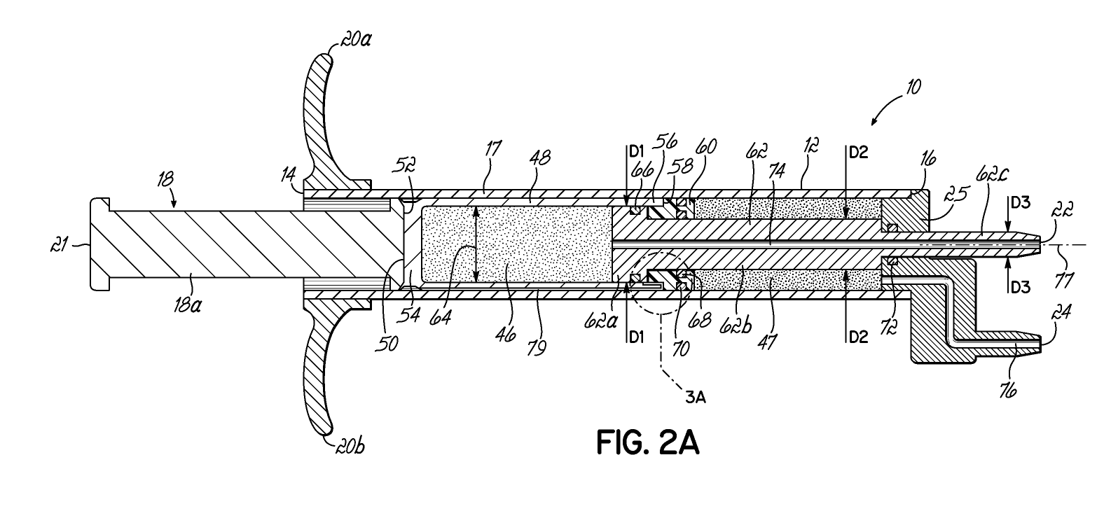

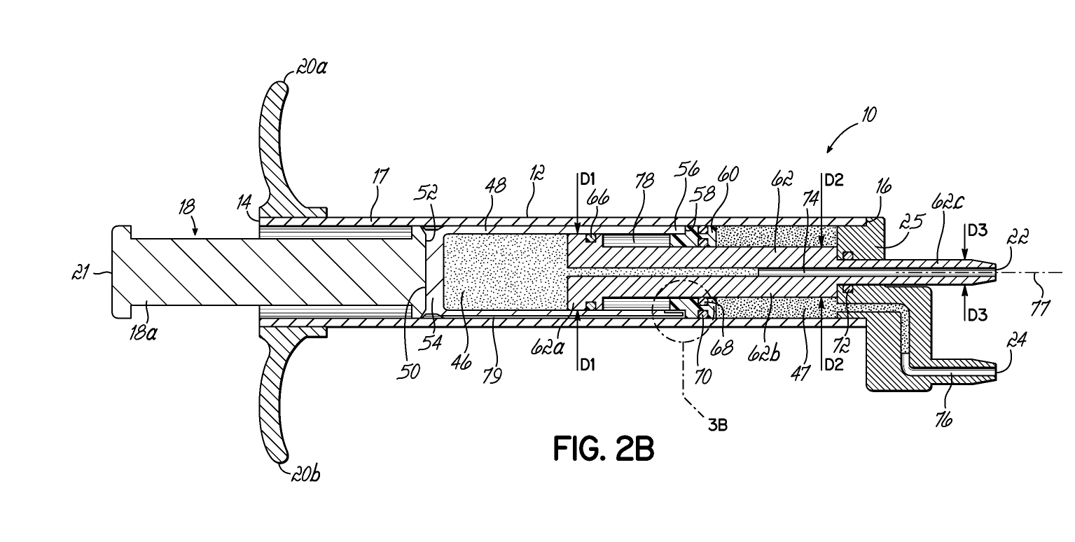

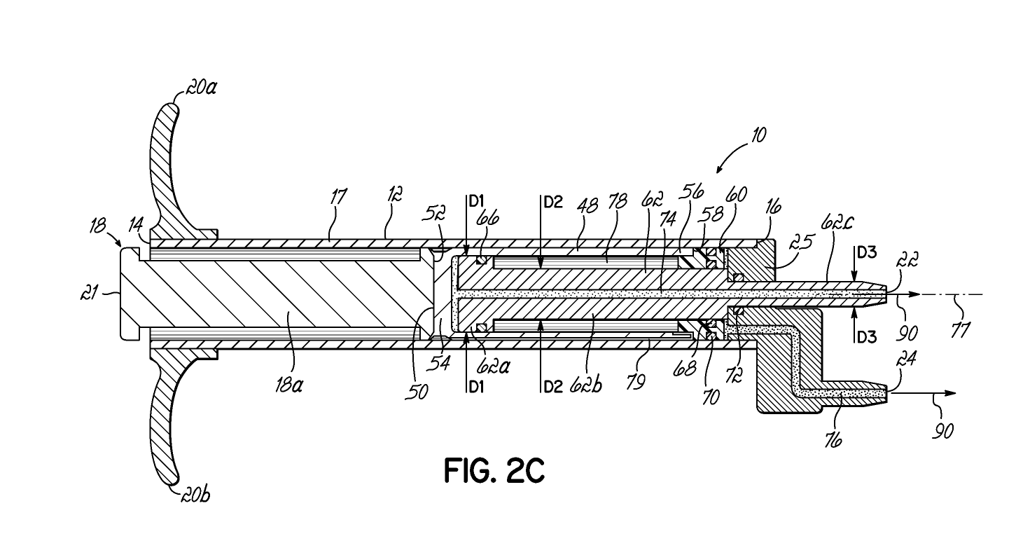

FIG. 2A shows the system 10 in a filled position. In a preferred embodiment, first and second fluid chambers 46, 47 are at least partially filled with first and second fluids. Preferably, transmission structure 18 is disposed within the barrel 12 at the proximal end 14, and the tip adaptor 25 is disposed within the barrel 12 at the distal end 16 prior to being packaged and sent to a clinical environment for use. The tip adaptor 25 closes and creates a seal at the distal end 16. Tip adaptor may be fixedly coupled to the distal end 16 of barrel 12 by various methods of manufacture known to persons skilled in the art. Preferably, the tip adaptor 25 is bonded to barrel 12 using an adhesive. Preferably, first and second fluid chambers 46, 47 are pre-filled with first and second fluids before being packaged and sent to a clinical environment for use. The system, however, may be packaged as unassembled and can be assembled and filled with first and second fluids on-site.

First piston 48 has a contact surface 50 at a first end 54 for accepting the transmission structure 18. The contact surface 50 and the contacting portion 52 of the transmission structure 18 contacting the contact surface 50 may have complimentary shapes for efficiency and ergonomics purposes. The first piston 48 may have a hollow, generally cylindrically shaped body extending distally from the contact surface 50 for forming part of the first fluid chamber 46. The second end 56 of the first piston 48 is open and includes a contact surface 58 that engages with the second piston 60. More specifically, the second end 56 is bonded to the second piston 60 generally at the location of the second end 56, such as by welding.

The delivery tube 62 may be fixedly, concentrically disposed relative to and within the barrel 12. In a preferred embodiment, the delivery tube 62 is coupled with the tip adaptor 25, thereby preventing the movement of delivery tube 62. The delivery tube may be fixed within the tip adaptor according to methods known to persons skilled in the art, such as sonic welding or bonding using adhesives. However, the manner of coupling is not limited to these methods and may be done so in other manners known to persons skilled in the art. The delivery tube 62 may have first, second and third sections 62a, 62b, 62c having first, second and third diameters D1, D2 and D3, respectively. The third section 62c may have a tapered portion at or near the first outlet 22 to enable the third section 62c to engage with a downstream device such as tubing or a cannula assembly 26 (see FIG. 1). Preferably, the first diameter D1 is larger than the second D2 and third D3, and the second diameter D2 is larger than the third diameter D3. In alternative embodiments, the first, second, and third diameters D1, D2 and D3 may vary, especially where the volume ratio of the first and second fluids are changed (as discussed below). The delivery tube 62 includes a first channel 74 disposed along the length and concentrically relative to first, second and third sections 62a, 62b, 62c.

The first diameter D1 and the inner diameter 64 of first piston 48 may be substantially the same, thereby creating a seal between the first piston 48 and delivery tube 62. In order to create an effective seal, a first O-ring 66 may be disposed on the delivery tube 62. The seal created by the first O-ring 66 may prevent the first fluid from the first chamber 46 to leak into other parts of the barrel 12, such as the cavity 78 that forms as the first and second pistons 48, 60 move distally. See FIGS. 2B, 2C. In an unengaged position, as seen in FIG. 2A, the first end 54 of the first piston 48 is proximal to the delivery tube 62, thereby defining a first fluid chamber 46 between the back wall 56 and the first section 62a of the delivery tube 62. The second piston 60 is at least partially disposed between the wall 17 of the barrel 12 and the second section 62b of the delivery tube 62. The second piston 60 has second and third O-rings 68, 70 disposed thereon. The second O-ring 68 provides a seal between the second section 62b of the delivery tube 62 and the second piston 60. The third O-ring 70 provides a seal between the second piston 60 and the wall 17 of the barrel 12. The seals provided by the second and third O-rings 68, 70 prevent the first fluid from escaping the first chamber 46 and entering other parts of the barrel 12, such as the cavity 78.

In the unengaged or filled state as seen in FIG. 2A, the transmission structure 18 may be pushed to cause distal movement of first piston 48 and thereby distal movement of the second piston 60 as well. The third section 62c of the delivery tube 62 is concentrically disposed relative to and passes through an aperture in the tip adaptor 25. To provide for an effective seal, there is a fourth O-ring 72 disposed on the tip adaptor 25 concentrically relative to the third section 62c of the delivery tube 62. The fourth O-ring 72 contacts the third section 62c of the delivery tube 62 and creates a seal, thereby preventing the second fluid from leaving the second fluid chamber 47 in an unwanted manner, such as through the aperture in the tip adaptor 25. Tip adaptor 25, second piston 60, wall 17 and delivery tube 62 define the boundaries for the second fluid chamber 47. The second fluid chamber 47 fluidicly connects with the second outlet 24 by way of the second channel 76. In one embodiment, at least one of the first or second outlets 22, 24 is disposed radially outward relative to a central axis 77 of the barrel. For example, in the embodiment shown in FIGS. 2A-B, the second outlet 24 is disposed radially outward from the central axis 77 of the barrel while the first outlet 22 is disposed along the axis 77.

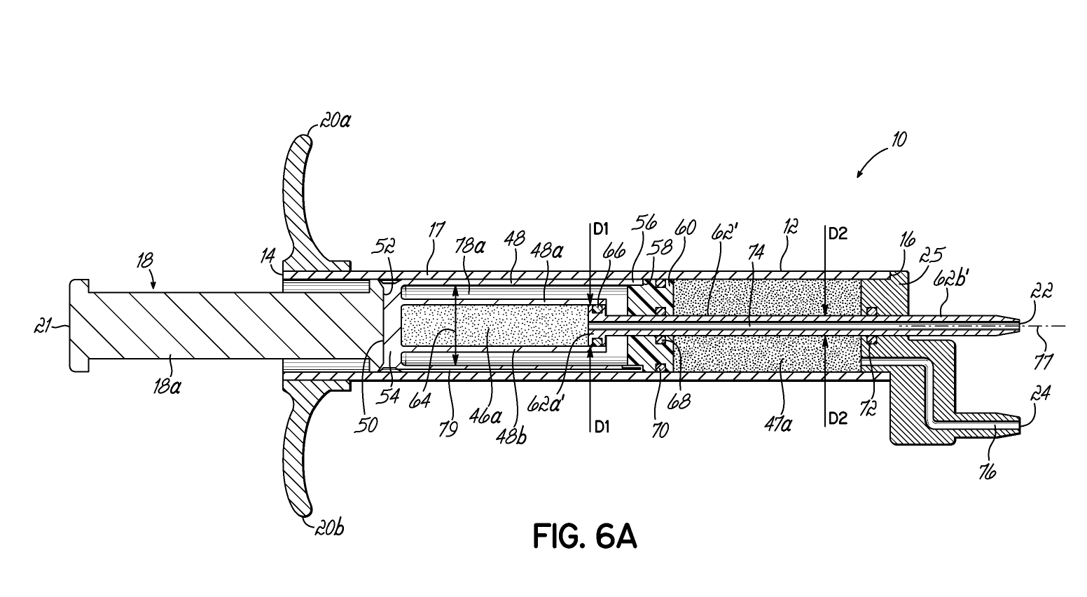

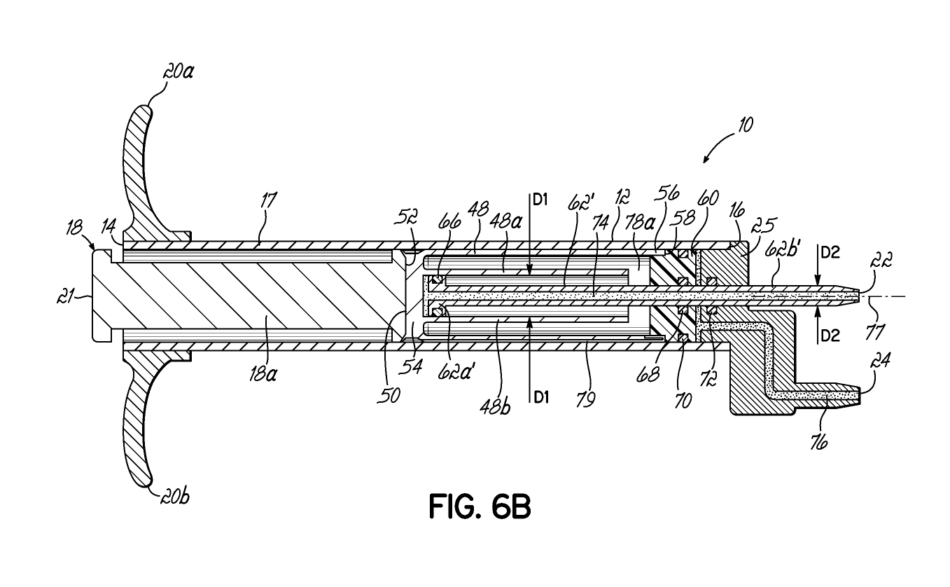

FIG. 2B shows the system in a position between the filled position and an empty position. Upon the distal movement of the transmission structure 18, the first end 54 of the first piston 48 is engaged with the transmission structure 18. The movement and engagement of the first piston 48 and the transmission structure 18 causes the distal movement of second piston 60. Due to the seal between the first piston 48 and the delivery tube 62 and the fixed nature of the delivery tube 62 relative to the movement of the first and second pistons 48, 60, the distal movement of the first piston 48 causes the first fluid to leave the first chamber 46 and enter into the first channel 74 and out of first outlet 22. The distal movement of first piston 48 causes the distal movement of the second piston 60, thereby causing the second fluid to leave the second chamber 47 and enter into the second channel 76 and out of the second outlet 24. In a preferred embodiment, the first and second fluids are dispensed out of the first and second outlets 22, 24 in a 1:1 ratio. However, in other embodiments, the ratio may be different, such as 2:1, 5:1, 10:1, and so on (see FIG. 6). FIG. 2C shows the system in a generally empty position after the first and second pistons 48, 60 have been advanced substantially distally.

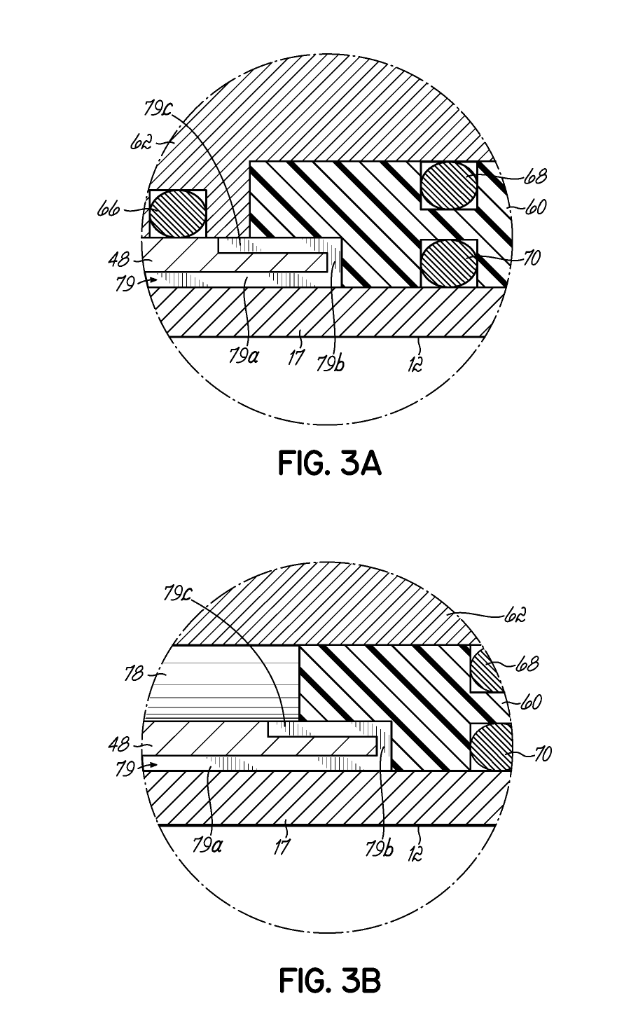

Cavity 78 increases in length as first and second pistons 48, 60 advance distally. Due to the sealed nature and configuration of the system, the cavity 78 has a natural tendency to develop a negative pressure, or become a vacuum space. To counteract the formation of a vacuum in cavity 78, a vent 79 is provided. In a preferred embodiment, vent 79 is configured as a channel along at least a portion of the length of the first piston 48, thereby preventing a vacuum from forming in cavity 78. FIG. 3A shows the vent 79 when the system 10 is in the filled position. Before the transmission structure 18, first piston 48 and second piston 60 advance distally, second piston 60 and delivery tube 62 may be in contact at the proximal end of the second piston 60. Vent 79 is formed as a channel on or within first piston 48 and fluidicly communicates with cavity 78 that forms between the second piston 60 and delivery tube 62 as the second piston 60 moves distally. In a preferred embodiment, the vent 79 is formed from a channel formed along at least a portion of the length of the first piston 48 and has first, second and third sections 79a, 79b, 79c. The first section 79a of the vent 79 is disposed between the first piston 48 and the wall 17 of the barrel 12. The second section 79b of the vent 79 extends radially inward and the third section 79c extends proximally away from the second section 79b and is generally parallel with the first section 79a. In an alternative embodiment, the vent 79 could be configured in any alternative manner that would counteract pressure changes in the cavity 78. As shown in FIGS. 2B, 2C and 3B, as the second piston 60 moves distally with respect to the delivery tube 62, the fluidic connection between the vent 79 and cavity 78 allows air to be drawn into cavity 78 from a space outside the system, thereby preventing negative pressure, or a vacuum, from forming in cavity 79.

Lubricious coatings may need to be provided between and among components due to the seals provided by the O-rings 66, 68, 70, 72. For example, to allow the traversal of the first and second pistons 48, 60 within the barrel 12 and past or along the delivery tube 62, lubricious coatings may be provided on the O-rings themselves or on the components with which they come into contact. For the same purpose, a lubricious coating may be provided on the transmission structure 18 and/or the barrel 12 for the traversal of the transmission structure 18 within the barrel 12.

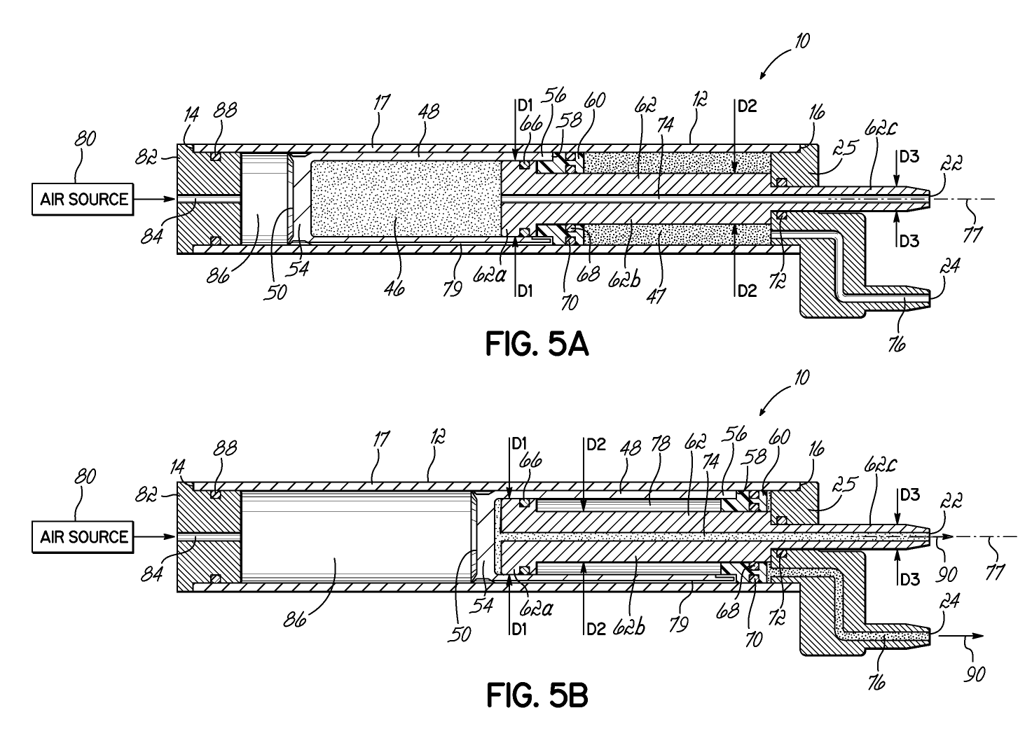

FIGS. 4, 5A and 5B show an alternative embodiment where the transmission structure 18 is a pneumatic air source 80. In this embodiment, the proximal end 14 of the barrel 12 is sealed by a pneumatic adaptor 82 having a bore 84 fluidicly communicating with air chamber 86. A fifth o-ring 88 may be provided in this embodiment to create a seal between the pneumatic adaptor 82 and the barrel 12. As seen in FIG. 5A, as air from the air source 80 is pumped into air chamber 86, the pressure within air chamber 86 increases, thereby causing the distal movement of the first piston 48 and increasing the length of the air chamber 86. The movement of the first piston 48 causes the distal movement of second piston 60. Due to the seal between the first piston 48 and the delivery tube 62 and the fixed nature of the delivery tube 62 relative to the movement of the first and second pistons 48, 60, the distal movement of the first piston 48 causes the first fluid to leave the first chamber 46 and enter into the first channel 74 and out of first outlet 22. The distal movement of first piston 48 causes the distal movement of the second piston 60, thereby causing the second fluid to leave the second chamber 47 and enter into the second channel 76 and out of the second outlet 24 in the direction of arrows 90. FIG. 5B shows the piston system in a plunged state after the first and second pistons 48, 60 have advanced distally.

As shown in FIG. 6A and 6B, in some embodiments, the ratio between the volumes of the first and second chambers 46a, 47a may be different. In FIGS. 6A and 6B, the ratio of fluid volume in the first and second chambers 46a and 47a is 11:1. This ratio is particularly advantageous in applications using blood-based biomaterials. In other embodiments, however, the ratio of volume between the first and second chambers 46a, 47a may be different, depending on the specific application and fluids. In one embodiment, the first chamber 46a contains a clotting agent, such as thrombin. In the second chamber 47a may be blood or a blood-based material without clotting agents therein.

In the embodiment shown in FIGS. 6A and 6B, delivery tube 62′ includes first and second sections 62a′, 62b′ having first and second diameters D1 and D2, respectively. First end 54 of the first piston 54, the legs 48a, 48b of the first piston 48, and the first section 62a′ of the delivery tube 62′ define the first chamber 46a. Similar to previously disclosed embodiments, when the transmission structure 18 moves distally, the first end 54 of the first piston 48 engages with the transmission structure 18. The movement and engagement of the first piston 48 and the transmission structure 18 causes the distal movement of second piston 60. Due to the seal between the legs 48a, 48b of first piston 48 and the delivery tube 62′, and also due to the fixed nature of the delivery tube 62′ relative to the first and second pistons 48, 60, the distal movement of the first piston 48 causes the first fluid to leave the first chamber 46 and enter into the first channel 74 and out of first outlet 22. The distal movement of first piston 48 causes the distal movement of the second piston 60, thereby causing the second fluid to leave the second chamber 47 and enter into the second channel 76 and out of the second outlet 24. In a preferred embodiment, the first and second fluids are dispensed out of the first and second outlets 22, 24. In one embodiment, the fluids may be mixed after being dispensed out of the first and second outlets 22, 24. Because the blood or blood-based material tends to clot when mixed with a clotting agent such as thrombin, the mixture of the first and second fluids may be used as a tissue barrier when mixed upon exiting the first and second outlets 22, 24. However, the embodiment shown in FIG. 6A and 6B is not limited to applications using blood or blood-based material and could be used in any application where different ratios of multiple fluids are desired.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method and illustrative examples shown and described. Accordingly, departures may be from such details without departing from the scope or spirit of the general inventive concept. What is claimed is:

1. A system for storing and dispensing first and second fluids, comprising:
a cartridge, further comprising:
a barrel having a wall, a proximal end and a distal end;
a first piston disposed within the wall, extending distally from the proximal end,
a delivery tube disposed within the wall and fluidicly communicating with a first outlet;
a first fluid chamber disposed between the first piston and the delivery tube, the first fluid chamber fluidicly communicating with the first outlet;
a second piston disposed between the wall and the delivery tube;
a second fluid chamber disposed between the second piston and the distal end, the second fluid chamber fluidicly communicating with a second outlet;
a transmission structure operative to transmit force from the first piston to the second piston to thereby dispense the first and second fluids from the first and second outlets; and
a cannula assembly coupled with and fluidicly communicating with the first and second outlets and further comprising:
a tip at a distal portion of the cannula assembly, adapted to dispense the first and second fluids from the cannula assembly.

2. The system of claim 1, wherein the distal end further comprises a tip adaptor, at least part of the second outlet disposed within and extending distally from the tip adaptor.

3. The system of claim 2, wherein at least one of the first or second outlets is disposed radially outward of a central axis of the barrel.

4. The system of claim 1, wherein the transmission structure comprises a plunger.

5. The system of claim 1, wherein the transmission structure is pneumatic.

6. The system of claim 1, wherein the cannula assembly further comprises:
first and second tubes having first and second lumens therein, respectively, the first and second tubes configured to be fluidicly coupled with the first and second outlets; and
the tip further comprises first and second apertures fluidicly communicating with the first and second lumens.

7. The system of claim 6, where the first and second tubes converge into a main cannula at a point distal to the first and second outlets, wherein the first and second fluids remain segregated within the main cannula as they traverse the first and second lumens.

8. The system of claim 1, further comprising a vent operative to prevent a vacuum from forming within the barrel.

9. The system of claim 8, wherein at least part of the vent is disposed between the first piston and the outer wall, the vent operative to prevent a vacuum from forming between the delivery tube and the second piston.

10. The system of claim 1 wherein the first and second fluids comprise different viscosities.

11. The system of claim 1, wherein the first and second fluids are reactive to form a tissue barrier upon an interaction of the first and second fluids.

12. The system of claim 10, wherein the first and second fluids are blood and a clotting agent.

13. The system of claim 12, wherein the clotting agent is thrombin.

14. A cartridge for storing and dispensing first and second fluids, comprising:
a barrel having a wall, a proximal end and a distal end;
a first piston disposed within the wall, extending distally from the proximal end;
a delivery tube disposed within the wall and fluidicly communicating with a first outlet;
a first fluid chamber disposed between the first piston and the delivery tube, the first fluid chamber fluidicly communicating with the first outlet;
a second piston disposed between the wall and the delivery tube;
a second fluid chamber between the second piston and the distal end, the second fluid chamber fluidicly communicating with a second outlet; and
a vent operative to prevent a vacuum from forming within the barrel;
wherein at least part of the vent is disposed between the first piston and the wall.

15. The cartridge of claim 13, wherein the vent is operative to prevent a vacuum from forming between the delivery tube and the second piston.

16. A method of counteracting pressure changes within a cartridge for storing and dispensing fluids, comprising:
advancing a plunger within a barrel, wherein advancing the plunger moves at least a first internal component in the barrel relative to at least a second internal component, thereby forming a cavity therebetween; and
fluidicly communicating the cavity with a space outside the cartridge.

17. The method of claim 15, wherein the cartridge includes a vent within the barrel.

18. The method of claim 16, wherein at least part of the vent is disposed between one of the internal components and a wall of the barrel.

19. A method of dispensing multiple fluids from a cartridge, the cartridge including a barrel, first and second pistons, first and second fluid chambers containing first and second fluids, a transmission structure, and first and second outlets, comprising:
fluidicly communicating the first and second outlets with first and second apertures of a cannula assembly;
advancing the transmission structure from a proximal end of the barrel towards a distal end, thereby advancing the first and second pistons within the barrel and discharging the first and second fluids from the first and second fluid chambers, through the first and second outlets, and out of first and second apertures at a distal end of the cannula assembly.

20. The method of claim 18, wherein the first fluid is blood and the second fluid is a clotting agent.

21. The method of claim 19, wherein the clotting agent is thrombin.

22. The method of claim 18 further comprising:
situating the first aperture at least partially transverse to the second aperture, whereby the first and second fluids are configured to interact after being discharged from the first and second apertures.

23. The method of claim 21 wherein an interaction between the first and second fluids creates a third material comprising the first and second fluids.

24. The method of claim 18, further comprising:
directing the first and second apertures towards a biological substrate, thereby dispensing the first and second fluids onto the biological substrate.

25. The method of claim 23, wherein the biological substrate is a bleeding anatomical site.

26. The method of claim 24, wherein the biological substrate comprises a plurality of anatomical sites, the fluids forming a tissue barrier between at least two of the anatomical sites.

27. A method of dispensing first and second biomaterial fluids from a cartridge, the cartridge including a barrel having a wall, a proximal end and a distal end, and axially disposed first and second chambers, the method comprising:
moving a first piston disposed within the wall of the barrel towards the distal end, the distal movement of the first piston causing the first biomaterial fluid to leave the first chamber and enter into a first channel and out a first outlet;
wherein the distal movement of the first piston distally moves the second piston, thereby causing the second biomaterial fluid to leave the second chamber and enter into a second channel and out of a second outlet;
wherein the first biomaterial fluid thereby passes from the first outlet to a first lumen of a cannula assembly and the second biomaterial fluid passes from the second outlet to a second lumen of the cannula assembly;
dispensing the first and second fluids from the cannula assembly by the aid of a pressurized gas;
mixing the first and second fluids in flight to form a mixture; and
depositing the mixture onto a portion of a human body.
